(19) United States
(12) Patent Application Publication
(10) Pub. No.: US 2018/0140312 A1
(43) Pub. Date: May 24, 2018

(54) DEVICES, APPARATUSES, KITS, AND METHODS FOR ALIGNMENT GUIDE AND FUSION DISK

(71) Applicant: Arthrosurface, Inc., Franklin, MA (US)

(72) Inventors: George SIKORA, Bridgewater, MA (US); Nikhil T. JAWRANI, Framingham, MA (US); Steven EK, JR., Bolton, MA (US)

(21) Appl. No.: 15/813,509

(22) Filed: Nov. 15, 2017

Related U.S. Application Data
(60) Provisional application No. 62/424,774, filed on Nov. 21, 2016.

(51) Int. Cl. A61B 17/17; A61B 17/80; A61B 17/16

(52) U.S. Cl. CPC A61B 17/1728; A61B 17/8033; A61B 2017/00477; A61B 17/1659; A61B 17/1739; A61B 17/1615

(57) ABSTRACT

Embodiments are disclosed that relate generally to orthopedic treatments, and more particularly, but not by way of limitation, to devices, apparatuses, kits, and methods for an orthopedic device comprising one or more alignment guides and/or fusion disks (e.g., for fusing together multiple bones at a fracture, injury, or surgery site).

### CROSS-REFERENCE TO RELATED APPLICATION

This application claims the benefit of U.S. Provisional Application No. 62/424,774, filed Nov. 21, 2016, the contents of which application is incorporated herein by reference in its entirety.

### BACKGROUND

### 1. Field of the Invention

The present invention relates generally to orthopedic treatments, and more particularly, but not by way of limitation, to devices, apparatuses, kits, and methods for an orthopedic device comprising one or more alignment guides and/or fusion disks (e.g., for fusing together multiple bones or pieces of bone, such as, for example, at a fracture or surgery site).

### 2. Description of Related Art

Examples of orthopedic alignment guides and/or fusion disks that can be used for a fusion procedure are disclosed in (1) U.S. Pat. No. 7,537,603, and (2) U.S. Pat. No. 6,179,839.

### SUMMARY

This disclosure includes embodiments of devices, apparatuses, kits, and methods for an orthopedic device comprising one or more alignment guides and/or fusion disks (e.g., for fusing together multiple bones at a fracture or surgery site).

Some embodiments of the present guide apparatuses comprise: a guide body having an upper side and an opposing lower side, the body defining a guide pin hole extending through the upper and lower sides of the body and configured to receive a guide pin, the body further defining a plurality of guide holes extending through the upper and lower sides of the body and corresponding to the positions of fastener holes in an implant, and the body having a plurality of radiopaque markers corresponding to at least some of the guide holes; where the orientation of each of the radiopaque markers is substantially fixed relative to the body to indicate a position of a corresponding one of the guide holes; and where the body is configured to be temporarily coupled to a bone, via a guide pin extending through the guide pin hole, such that: (i) the lower side of the body faces the bone; (ii) the upper side of the body faces away from the bone; and (iii) the body is rotatable around the guide pin relative to the bone.

In some embodiments of the present apparatuses, the body has a vertical central axis and the plurality of guide holes each has a central axis that extends toward the vertical central axis (e.g., the vertical central axis of the body may extend through the center of the pin hole). In some embodiments, the upper side comprises a planar area delineated by a perimeter, and the lower side is convex and extends to the perimeter (e.g., circular perimeter). In some embodiments, the plurality of radiopaque markers are disposed on or in the body. In some embodiments, the plurality of radiopaque markers comprise a plurality of protrusions extending from the guide body.

In some embodiments of the present apparatuses, the plurality of radiopaque markers each have a proximal end, a distal end opposing the proximal end, an upper side, a lower side opposing the upper side, and a pair of lateral sides extending between the proximal end, the distal end, the upper side, and the lower side, a length extending between the proximal and distal ends, a height extending between the upper and lower sides, and a width extending between the opposing lateral sides. In some embodiments, the plurality of radiopaque markers form a unitary piece with the guide body. In some embodiments, the width of the distal end is greater than the width of the proximal end. In some embodiments, a surface of the distal end is curved or arcuate along the width of the distal end.

In some embodiments of the present apparatuses, the upper side of each radiopaque marker is disposed in a plane parallel to the lower side of each radiopaque marker. In some embodiments, the upper side and the lower side of each radiopaque marker are each disposed in a plane parallel to the upper side of the guide body.

In some embodiments of the present apparatuses, the lower side of each radiopaque marker is configured to abut an outer surface of the bone.

In some embodiments of the present apparatuses, each of the plurality of radiopaque markers corresponds to one of the plurality of guide holes, each guide hole being disposed between the vertical central axis of the body and its corresponding radiopaque marker. In some embodiments, each radiopaque marker has a central axis that lies along a radius between the vertical central axis of the body and the distal end, the central axis of each radiopaque marker passing through the central axis of its corresponding guide hole. In some embodiments, the radius between the vertical central axis of the body and the distal end is greater than a radius between the vertical central axis of the body and the perimeter of the guide body. In some embodiments, each of the plurality of radiopaque markers comprises a guide label that identifies its corresponding one of the plurality of guide holes.

In some embodiments of the present apparatuses, the plurality of guide holes are each configured to receive one of a drill mechanism and a guide pin into each guide hole.

In some embodiments of the present apparatuses, the body is configured to be temporarily coupled to the bone by one or more guide pins extending through one or more of the plurality of guide holes into the bone.

In some embodiments of the present apparatuses, the central axis of each of the plurality of guide holes intersects the vertical central axis of the body at a point located at a distance closer to the upper side of the body than the lower side of the body.

In some embodiments of the present apparatuses, the guide pin hole is disposed at a center of the body and extends through the upper side and lower side of the body. In some embodiments, a central axis of the guide pin hole corresponds to the vertical central axis of the body, and the body is configured to be temporarily coupled to the bone by the guide pin extending through the guide pin hole into the bone.

In some embodiments of the present apparatuses, the guide body comprises a radiopaque material.

Some embodiments of the present kits comprise: an embodiment of the present guide apparatuses; and a package within which the guide apparatus is sealed. Some embodiments further comprise one or more guide pins.

Some embodiments of the present bone-implant apparatuses comprise: an implant body having an upper side comprising a planar area and a lower side comprising a convex area, the lower side opposing the upper side and meeting the upper side at a perimeter, the body defining a plurality of fastener holes extending through the upper side and lower side of the body; where the body is configured to be coupled to a bone such that: (i) the lower side of the body faces the bone; and (ii) the upper side of the body faces away from the bone. In some embodiments, the body has a vertical central axis and the plurality of fastener holes each has a central axis that extends toward the vertical central axis. In some embodiments, the upper side and the lower side are circular and the perimeter is a circumferential perimeter. In some embodiments, the plurality of fastener holes are each configured to receive one of a guide pin and a fastener along the respective central axis. In some embodiments, the central axis of each of the plurality of fastener holes intersects the vertical central axis of the body. In some embodiments, the body is configured to be temporarily coupled to the bone by one or more guide pins extending through one or more of the plurality of fastener holes into the bone. In some embodiments, the body is configured to be coupled to the bone by one or more fasteners extending through one or more of the plurality of fastener holes into the bone. In some embodiments, the body further comprises a guide pin hole disposed at a center of the body and extending through the upper side and lower side of the body, where a central axis of the guide pin hole corresponds to the vertical central axis of the body, and where the body is configured to be temporarily coupled to the bone by the guide pin extending through the guide pin hole into the bone. In some embodiments, the implant body comprises a metal, a plastic, an allograft material, a xenograft material, or a composite material.

Some embodiments of the present kits comprise: an embodiment of the present bone-implant apparatuses; and a package within which the cone-implant apparatus is sealed. Some embodiments further comprise: one or more guide pins and/or one or more fasteners.

Some embodiments of the present methods (e.g., of modifying a bone) comprise: forming a recess in the bone with a cutting device; positioning the lower side of the guide body of one of the present guide apparatuses to abut a bottom surface of the recess; positioning the plurality of radiopaque markers to a desired position; and inserting guide pins through one or more of the plurality of guide holes of the guide body into the bone.

Some embodiments of the present methods further comprise: inserting, before forming the recess, a guide pin into the bone in a position where the guide pin is configured to pass through a guide pin hole of the guide body.

Some embodiments of the present methods further comprise: inserting, before inserting the guide pins into the plurality of guide holes, a drill mechanism through one or more of the plurality of guide holes of the guide body; and drilling one or more holes into the bone.

Some embodiments of the present methods further comprise: removing the guide body from the guide pins while the guide pins are inserted into the bone.

Some embodiments of the present methods further comprise: disposing at least a portion of an embodiment of the present bone-implant apparatuses into the recess.

In some embodiments of the present methods, positioning the plurality of radiopaque markers to a desired position comprises positioning a plurality of radiopaque markers to abut an outer surface of the bone. In some embodiments, disposing the bone-implant apparatus into the recess comprises passing a guide pin through a guide pin hole of the bone-implant apparatus. In some embodiments, disposing the bone-implant apparatus into the recess comprises passing a guide pin through a fastener hole of the bone-implant apparatus.

Some embodiments of the present methods further comprise: inserting a fastener through each of the fastener holes of the bone-implant apparatus and into a hole in the bone. In some embodiments, the hole in the bone is drilled with the drilling apparatus. In some embodiments, each fastener extends into a separate bone.

In some embodiments of the present methods, the cutting device is a reamer having a body extending outwardly relative to a rotational axis, the body having a proximal cutting end, a distal end, and defining a hollow channel extending through the body from the proximal end to the distal end along the rotational axis. Some embodiments further comprise: disposing the reamer over the guide pin configured to pass through the guide pin hole of the guide body such that the guide pin extends through the hollow channel and the proximal cutting end is in contact with a surface of the bone. Some embodiments further comprise: rotating the reamer around the guide pin to form the recess in the bone; and removing the reamer from the guide pin.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present devices, apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device, or structure that is configured in a certain way is configured in at least that way, but it can also be configured in other ways than those specifically described.

Any embodiment of any of the present devices, apparatuses, kits, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiments depicted in the figures.

FIGS. 1A-1E depict various views of a first embodiment of the present alignment guide.

FIGS. 2A-2H depict various views of a first embodiment of the present alignment fusion disk.

FIGS. 3A-3H depict various views of a second embodiment of the present alignment fusion disk.

FIGS. 4A-4F depict various views of stages of the present methods using the alignment guide of FIGS. 1A-1E with the osteotomy fusion disk of FIGS. 3A-3H.

### DESCRIPTION OF ILLUSTRATIVE EMBODIMENTS

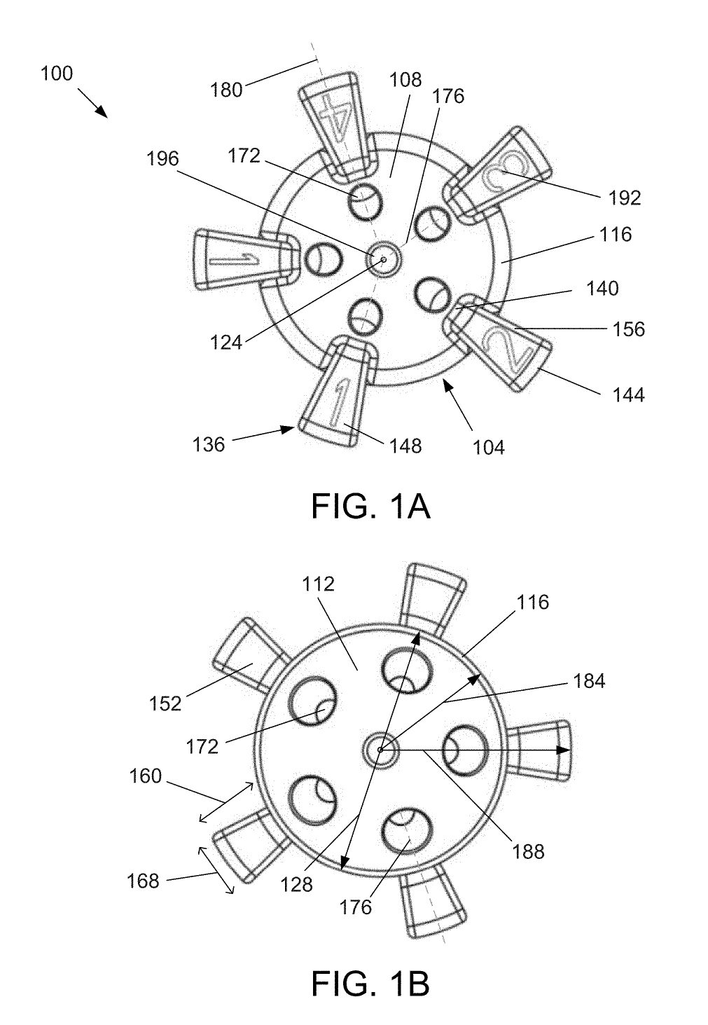

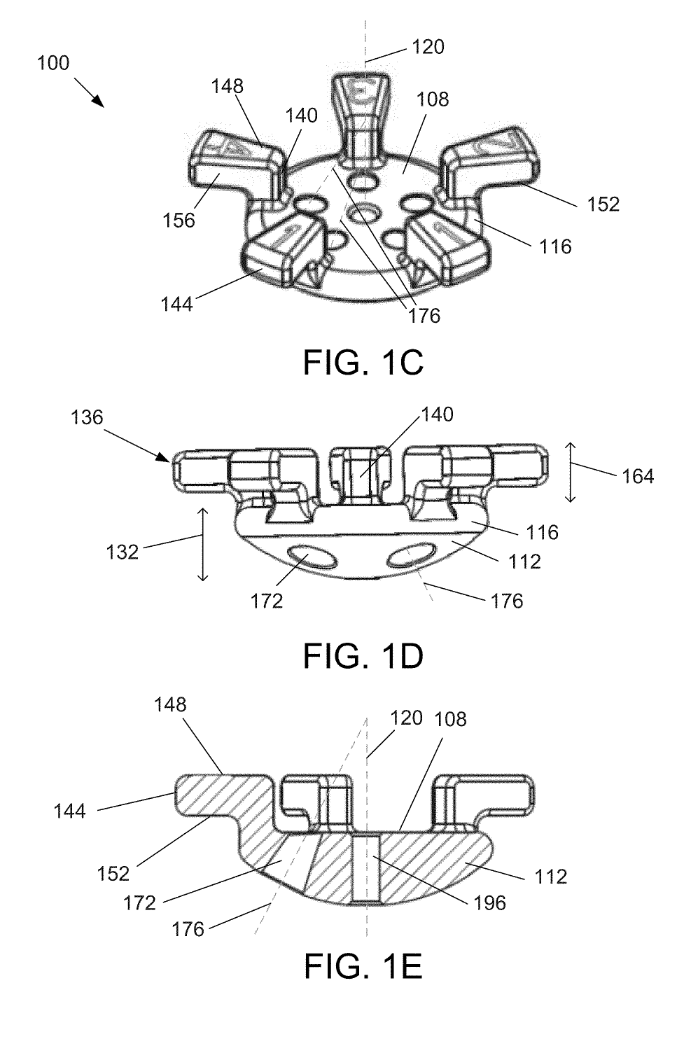

Referring now to the drawings, and more particularly to FIGS. 1A-1E, shown there and designated by the reference numeral 100 are various views of a first embodiment of the present alignment guide. In the embodiment shown, guide 100 has a guide body 104, an upper side 108, and an opposing lower side 112. As shown, guide body 104 has a perimeter 116 disposed between the upper side 108 and lower side 112, and upper side 108 meets lower side 112 at perimeter 116. In this embodiment, upper side 108 has a circular shape and perimeter 116 is circumferential. In the embodiment shown, upper side 108 is substantially planar (e.g., defines a planar area delineated by perimeter 116, as shown), and lower side 112 also has a circular shape having an area delineated by perimeter 116. As shown, lower side 112 is convex relative to the planar upper side 108. In other embodiments, upper side 108 and/or lower side 112 may have other suitable shapes (e.g., elliptical, ovoid) and respective areas delineated by other suitable perimeters 116. In the embodiment shown, body 104 has a central axis 120 that passes through center 124 of upper side 108 and lower side 112. As shown, central axis 120 is vertical (i.e., perpendicular to the plane defining the surface of upper side 108). Body 104 (and circular perimeter 116) has a diameter 128, and a height 132 that is greatest at central axis 120 and decreases as the surface of lower side 112 tapers toward perimeter 116. In some embodiments, diameter 128 is between 14 mm and 24 mm (e.g., 16 mm); and/or height 132 is between 3 mm and 12 mm (e.g., 5 mm). In the depicted embodiment, perimeter 116 (the portion of body 104 defining perimeter 116) is curved and/or arcuate (e.g., filleted) between upper side 108 and lower side 112. In other embodiments, perimeter 116 can have other suitable shapes (e.g., upper side 108 and lower side 112 can meet at an edge).

In the embodiment shown in FIGS. 1A-1E, body 104 also has a plurality of radiopaque markers 136. In this embodiment, radiopaque markers 136 comprise a plurality of protrusions. As shown, each marker 136 has a proximal end 140, an opposing distal end 144, an upper side 148, an opposing lower side 152, and a pair of opposing lateral sides 156 extending between proximal end 140 and distal end 144 and between upper side 148 and lower side 152. A length 160 of each marker 136 extends between proximal end 140 and distal end 144, a height 164 of each marker 136 extends between upper side 148 and lower side 152, and a width 168 of each marker 136 extends between opposing lateral sides 156. In the embodiment shown, each marker 136 is elongated with length 160 that is greater than its height 164 and/or its width 168. In this embodiment, height 164 at proximal end 140 is greater than height 164 at distal end 144, and width 168 at distal end 144 is greater than width 168 at proximal end 140. As shown, the surface of distal end 144 is curved or arcuate along the width 168 of distal end 144. In some embodiments, length 160 is between 4 mm and 16 mm (e.g., 8 mm); height 164 is between 1 mm and 6 mm (e.g., 3 mm); and/or width 168 is between 2 mm and 12 mm (e.g., 6 mm). In other embodiments, radiopaque markers 136 may comprise other suitable configurations, such as, for example, cylindrical projections with constant cross-sections along their respective lengths.

In the embodiment shown in FIG. 1A-1E, radiopaque markers 136 are coupled to and extend outward from guide body 104. In the depicted configuration, radiopaque markers 136 are each disposed on and extend upward from the upper side 108 of body 104, and also extend outward beyond perimeter 116. In this embodiment, markers 136 form a unitary piece with body 104 but, in other embodiments, markers 136 may be formed as separate pieces that are thereafter attached to body 104. As shown (e.g., in FIGS. 1D-1E), upper side 148 of each radiopaque markers 136 is disposed in a plane that is different than and disposed above (e.g., and parallel to, as shown) the plane of upper side 108 of body 104. Similarly, lower side 152 of each marker 136 is disposed in a plane that is different than and disposed below (e.g., and parallel to, as shown) the plane of upper side 108 of body 104, such as, for example, to provide clearance above tissues that may be adjacent to a bone surface on which body 104 may be disposed (e.g., to permit body 104 to be rotated in contact with the bone surface). In other embodiments, guide 100 may include radiopaque markers (in addition to, or in place of markers 136) that are disposed on a surface of or embedded within body 104; for example, such markers may include a radiopaque material (e.g., paint) disposed on upper side 108 and/or radiopaque members (e.g., metal rods or strips) embedded within body 104.

In the embodiment shown in FIGS. 1A-1E, body 104 defines a plurality of guide holes 172. In the embodiment shown, guide holes 172 are defined by body 104 and extend between upper side 108 and lower side 112 of body 104. Each guide hole 172 has a central axis 176, and corresponds to one of the radiopaque markers (e.g., 136). In other embodiments, other relative numbers of guide holes and markers may be used; for example, one marker between each two adjacent holes. In the embodiment shown, the orientation of each of the radiopaque markers 136 is substantially fixed relative to body 104 to indicate a position of a corresponding guide hole 172. In this embodiment, the position of each guide hole 172 in body 104 also corresponds to the position of a fastener hole of an implant corresponding to guide 100, as further described below. In the embodiment shown, there are five (5) guide holes 172 and five (5) corresponding radiopaque markers 136. Other numbers of guide holes 172 and corresponding radiopaque markers 136 can be used. In some embodiments, the number of guide holes 172 and corresponding radiopaque markers 136 is between three (3) and eight (8).

In the embodiment shown, each guide hole 172 in body 104 is disposed between central axis 120 and proximal end 140 of a corresponding radiopaque marker 136. In the embodiment shown, each marker 136 has a central axis 180 extend radially outward from central axis 120 through proximal end 140 and distal end 144 of the marker (136). As shown, in this configuration, central axis 180 of each guide hole 172 also intersects the central axis (176) of its corresponding guide hole 172. In the embodiment shown, radius 184 defined between central axis 120 and distal end 144 of each radiopaque marker 136 is greater than a radius 188 defined between central axis 120 and perimeter 116 of body 104. In this way, radiopaque markers 136 extend radially outwardly from central axis 120 and extend a distance past perimeter 116. In this embodiment, each marker 136 has a guide label 192 on its upper side 148 to denote a number or other reference to assist with aligning guide holes 172 and/or an implant corresponding to guide 100, as described in more detail below.

One or more of the central axes 176 of guide holes 172 can be angled toward central axis 120. For example, in the embodiment shown in FIGS. 1A-1E, each central axes 176 intersects central axis 120 at a point above upper side 108 of body 104. In this configuration, an upper end of each guide hole 172 is closer to central axis 120 than a corresponding lower end of each guide hole 172 such that guide holes 172 are angled relative to central axis 120. Each guide hole 172 is configured to receive a drill bit and/or a guide pin to align and/or define holes in underlying bone to receive screws and thereby secure a corresponding implant, as described below. In some embodiments, each guide hole 172 is configured to receive a drill bit and/or guide pin coaxial to its central axis 176. In other embodiments, one or more of the central axes 176 may extend in other directions (e.g., a direction perpendicular to the plane of upper side 108).

In the embodiment shown in FIGS. 1A-1E, guide body 104 also defines a guide pin hole 196 extending between and through upper side 108 and lower side 112. As shown, guide pin hole 196 is disposed at the center 124 of upper side 108 and lower side 112 is aligned with and centered on central axis 120. In this configuration, guide pin hole 196 extends perpendicular to the plane of upper side 108 of body 104. In other embodiments, additional guide pin holes 196 can be provided. In the embodiment shown, guide pin hole 196 is configured to receive a guide pin coaxial to central axis 120. In the embodiment shown, body 104 is rotatable around a guide pin and relative to a bone in which the guide pin is embedded. In some embodiments, body 104 comprises a radiopaque material. In some embodiments, body 104 comprises a material that is different than that of markers 136 (e.g., a second radiopaque material, or a material that is not radiopaque).

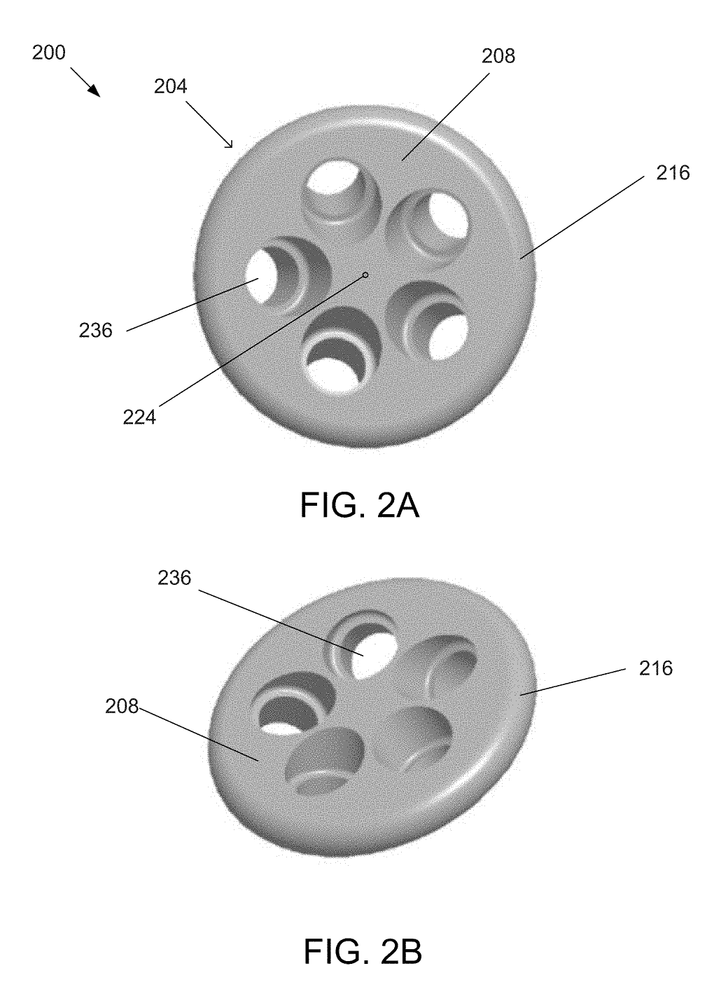

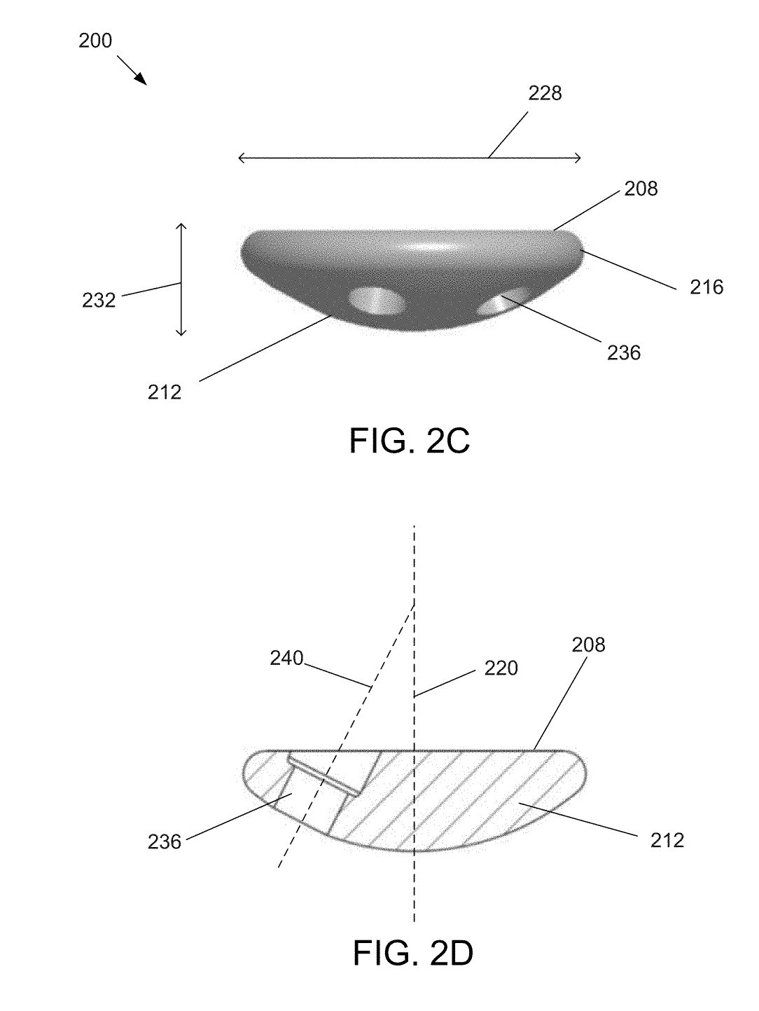

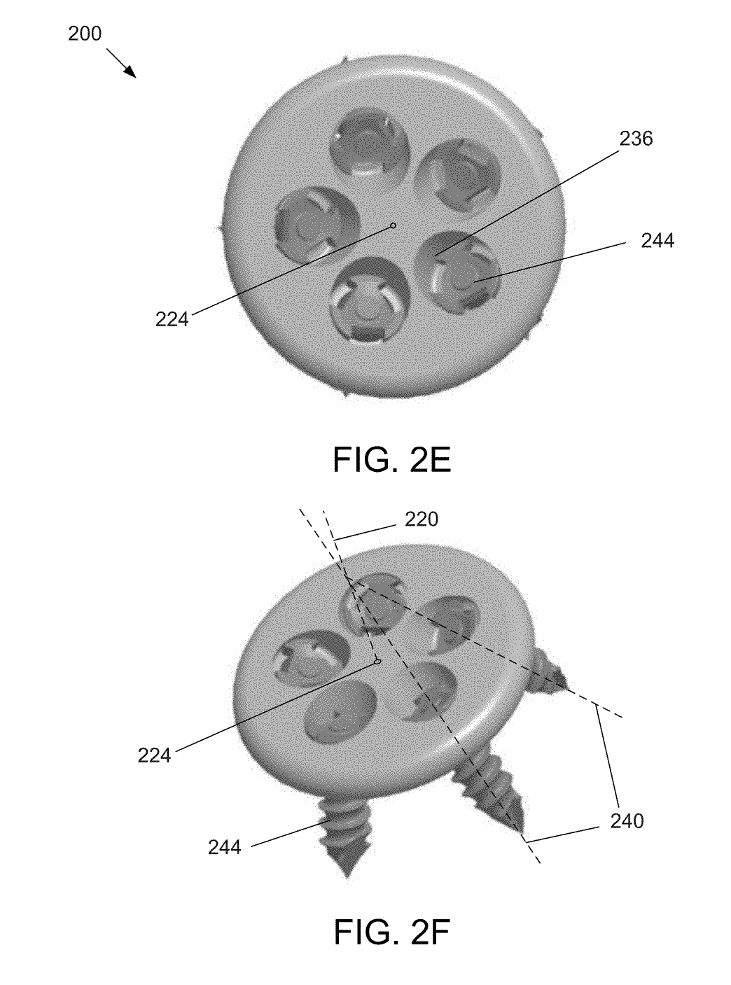

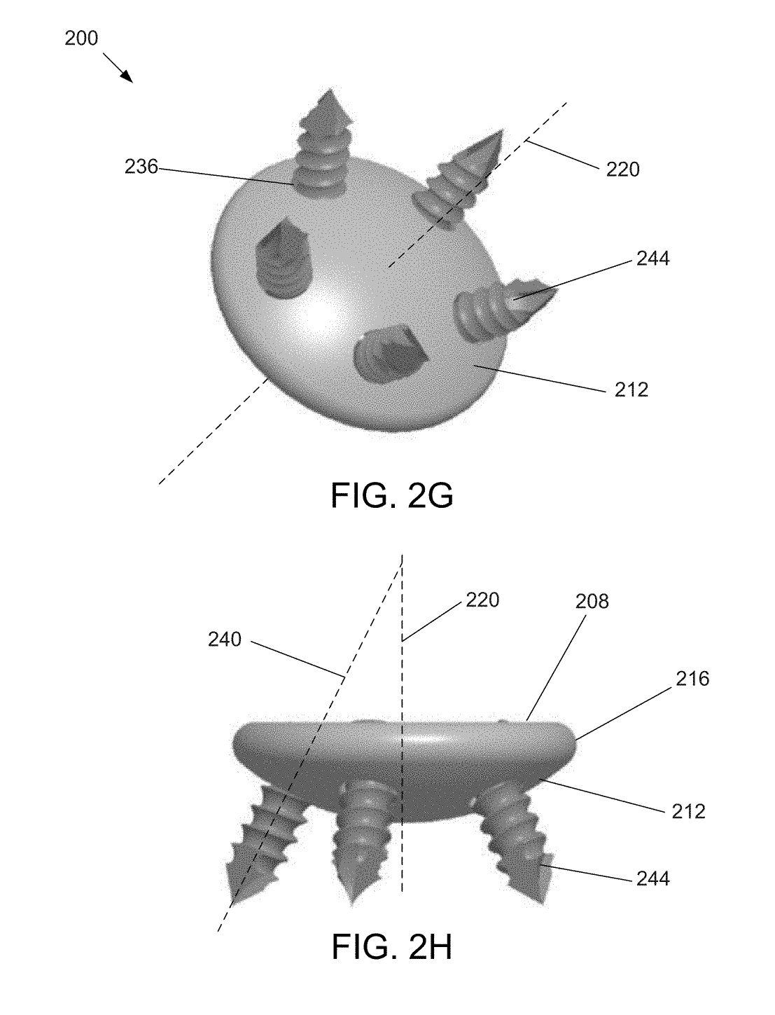

FIGS. 2A-2H depict various views of a first embodiment of the present implants configured for use with the guide of FIGS. 1A-1E. In the embodiment shown, disk 200 is a fusion disk implant. As shown, disk 200 has an implant body 204 with an upper side 208, an opposing lower side 212, and a perimeter 216 between the upper side 208 and lower side 212 and at which upper side 208 and lower side 212 meet. In this embodiment, upper side 208 is planar and has a circular shape. As shown, lower side 212 also has a circular shape that, together with the circular shape of the upper side, results in a circular or circumferential perimeter 216. In the embodiment shown, lower side 212 is convex relative to the plane of upper side 208. In other embodiments, upper side 208 and/or lower side 212 may have other suitable shapes (e.g., elliptical, ovoid) and may result the perimeter at which they meet having a non-circular shape. In some embodiments, implant body 204 is substantially similar in shape to guide body 104. In the embodiment shown, body 204 has a central axis 220 that passes through the center 224 of upper side 208 and lower side 212. As shown, central axis 220 is perpendicular to the plane of upper side 208. Body 204 (and circular perimeter 216) has a diameter 228, and a height 232 that is greatest at central axis 220 and decreases as the surface of lower side 212 tapers toward perimeter 216. In some embodiments, diameter 228 is between 14 mm and 24 mm (e.g., 16 mm); and/or height 232 is between 3 mm and 12 mm (e.g., 5 mm). In the depicted embodiment, perimeter 216 (the portion of body 204 defining perimeter 216) is curved and/or arcuate (e.g., filleted) between upper side 208 and lower side 212. In other embodiments, perimeter 216 can have other suitable shapes (e.g., upper side 208 and lower side 212 can meet at an edge).

In the embodiment shown in FIGS. 2A-2H, body 204 defines a plurality of fastener holes 236 extending between and through upper side 208 and lower side 212 of body 204. Each fastener hole 236 has a central axis 240. As with guide body 104, one or more of the central axes 240 of fastener holes 236 can be angled toward central axis 220. For example, in the embodiment shown in FIGS. 2A-2H, each central axis 240 intersects central axis 220 above upper side 208 of body 204. In this configuration, an upper end of each fastener hole 236 is closer to central axis 220 than a corresponding lower end of each fastener hole 236 such that fastener holes 236 are angled relative to central axis 220. Each fastener hole 236 is configured to receive a fastener 244 coaxial to its central axis 240. Fastener 244 can be a pin, screw, locking screw, or other suitable mechanism for securing disk 200 to a bone. In some embodiments, body 204 comprises a radiopaque material. In some embodiments, body 204 comprises an allograft material, a xenograft material, a metal, a plastic, and/or a composite material.

Ultimately, in the embodiments shown, the shape of implant body 204 closely corresponds to the shape of guide body 104, and the number and orientation of guide holes 172 in guide body 104 corresponds to the number and orientation of fastener holes 236 in implant body 204, such that guide 100 can be temporarily affixed to an implant site to orient fastener holes in the bone to accept fasteners through the fastener holes (236) of the implant body (204).

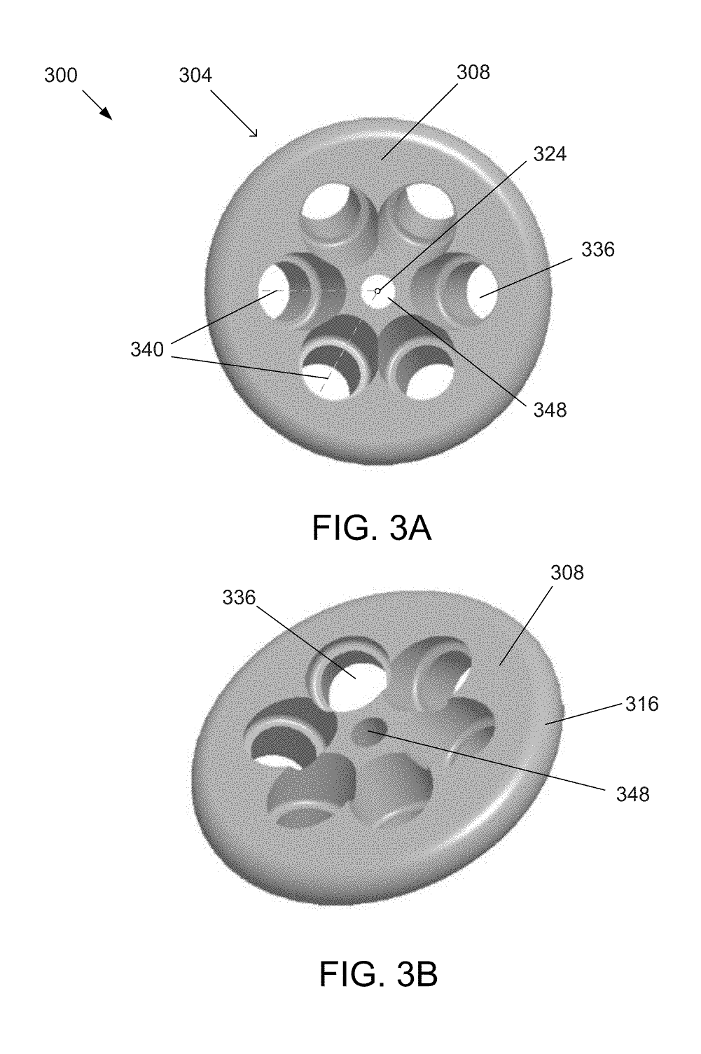

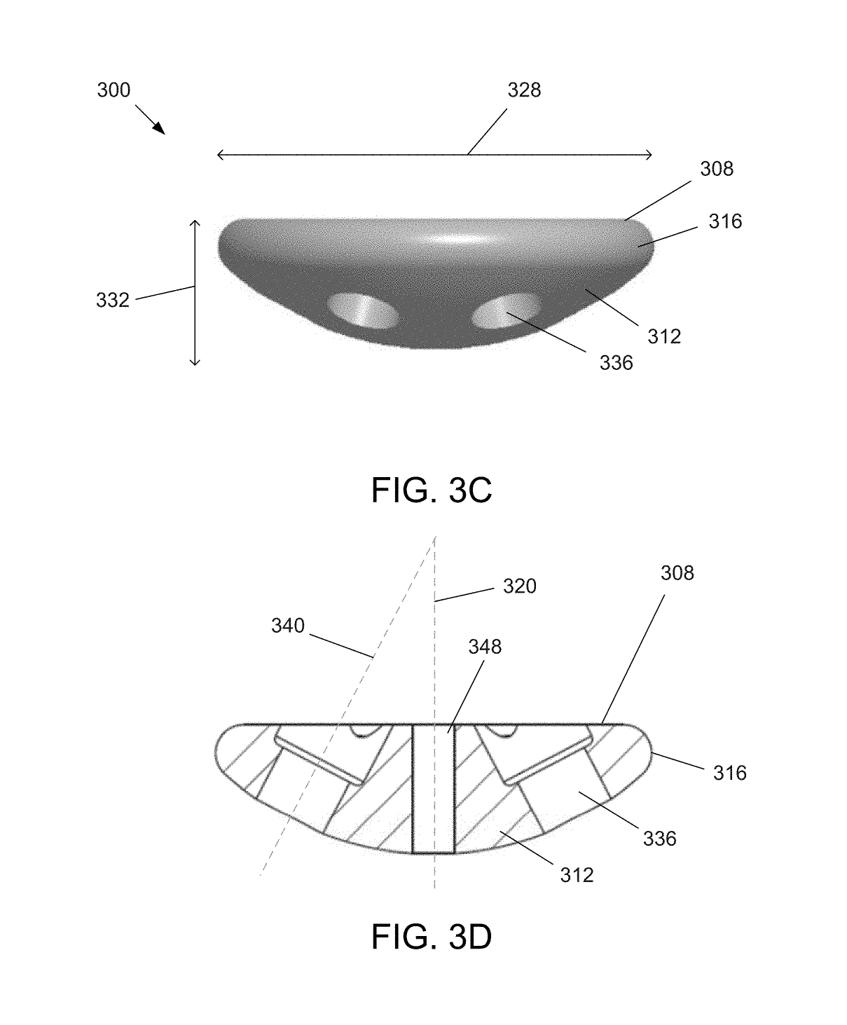

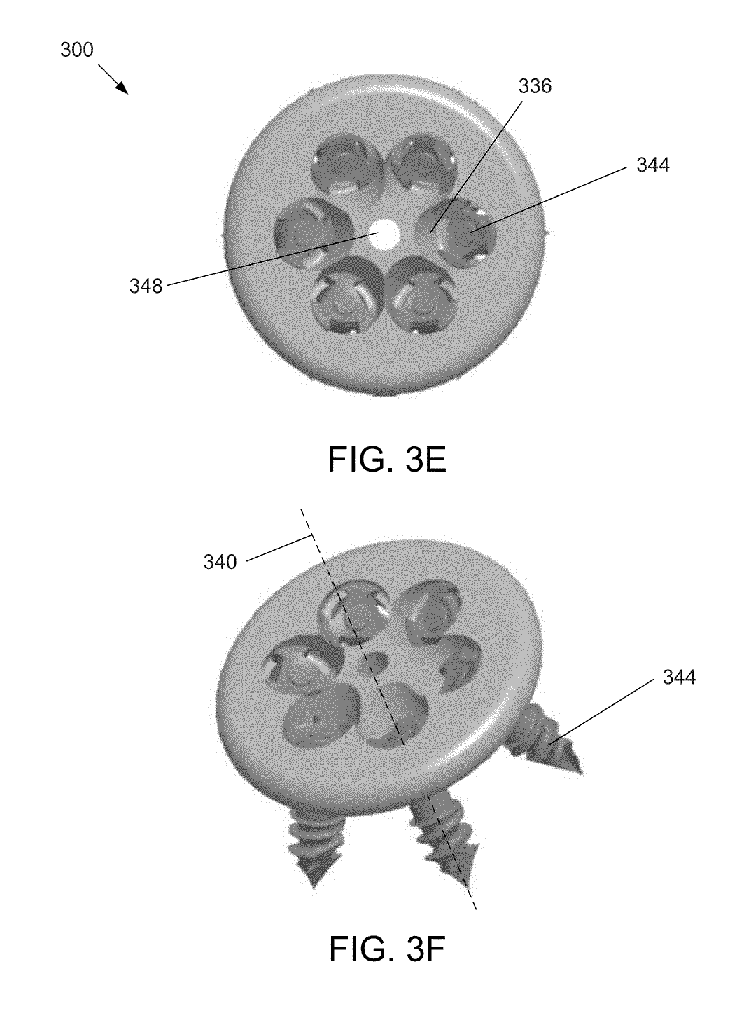

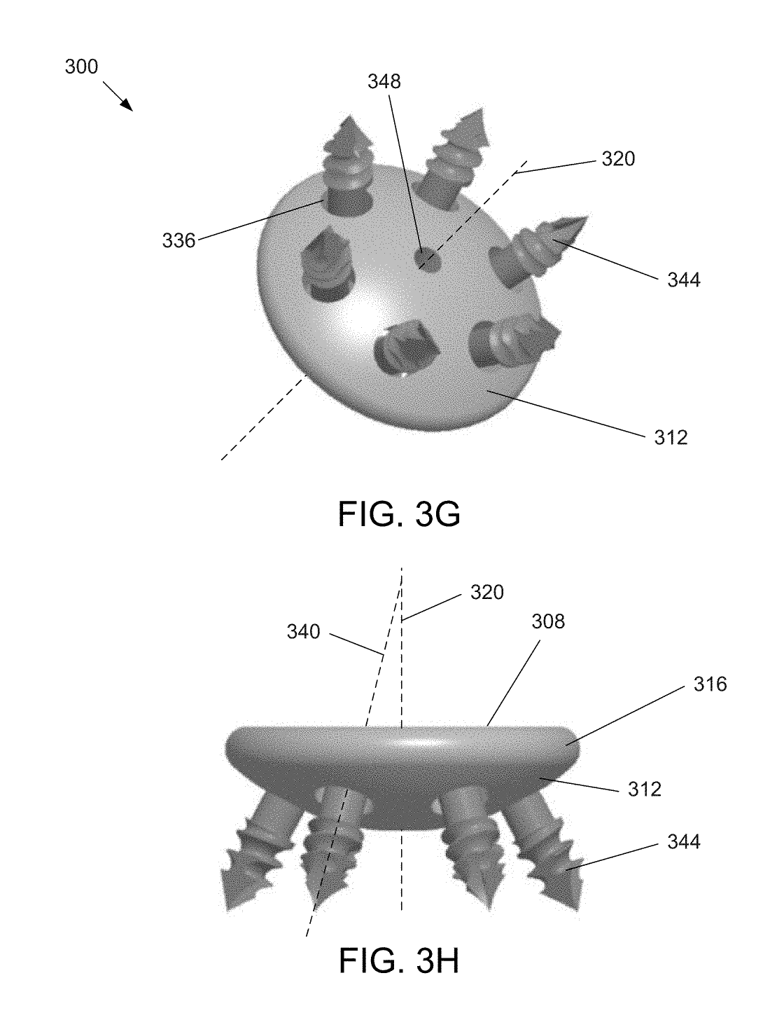

FIGS. 3A-3H depict various views of a second embodiment 300 of the present implants configured for use with the guide 100 of FIGS. 1A-1E. Implant 300 is substantially similar to implant 200. For example, implant 300 has body 304, an upper side 308, a lower side 312, a perimeter 316, a central axis 320, a center 324, a diameter 328, a height 332, and fastener holes 336 with central axes 340 that are similar in many respects to the corresponding features of implant 200. However, implant 300 does differ relative to implant 200 in some ways. Specifically, body 304 of implant 300 includes a guide pin hole 348 and body 304 defines six (6) fastener holes 336 whereas body 204 defines only five (5) fastener holes 236. In other embodiments, different numbers of fastener holes 236, 336, can be provided, with a matching guide 100 having an equal number of guide holes 172 and radiopaque markers 136 disposed in the same orientation as implants 200, 300.

Guide pin hole 348 extends between and through upper side 308 and lower side 312. As shown, guide pin hole 348 is disposed at the center 324 of upper side 308 and lower side 312, and is aligned with and centered on central axis 320. In this configuration, guide pin hole 348 extends perpendicular to the plane of upper side 308 of body 304. In other embodiments, additional guide pin holes 348 can be provided. In the embodiment shown, guide pin hole 348 is configured to receive a guide pin coaxial to central axis 320.

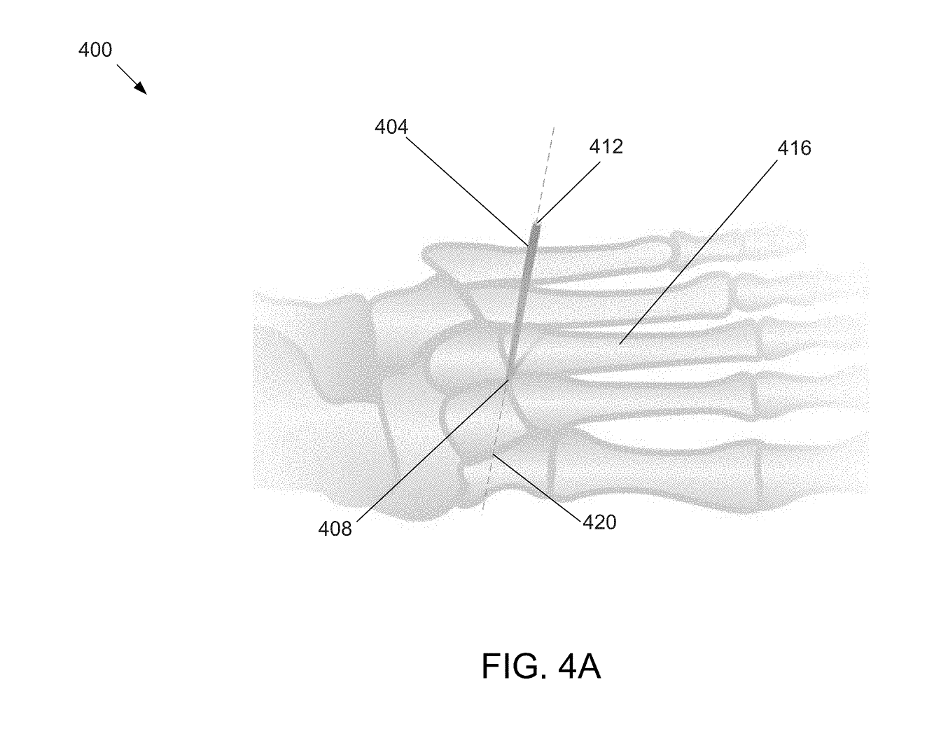

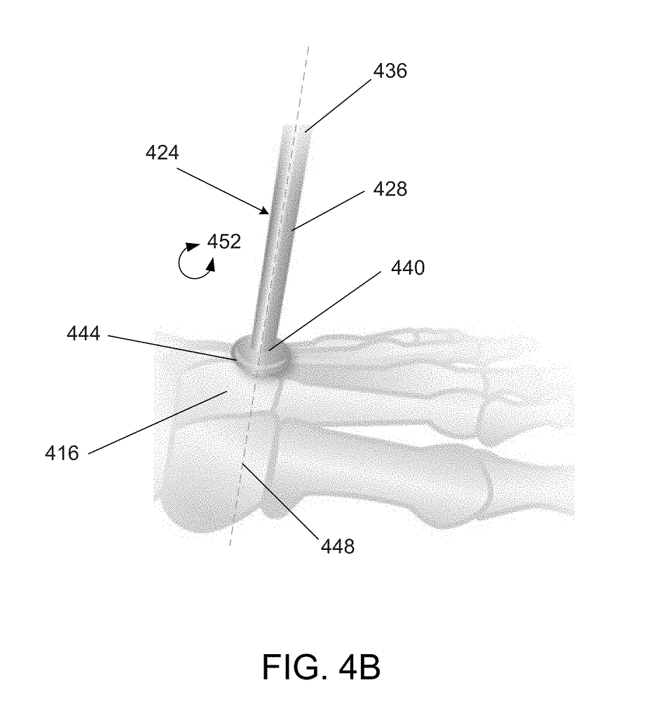

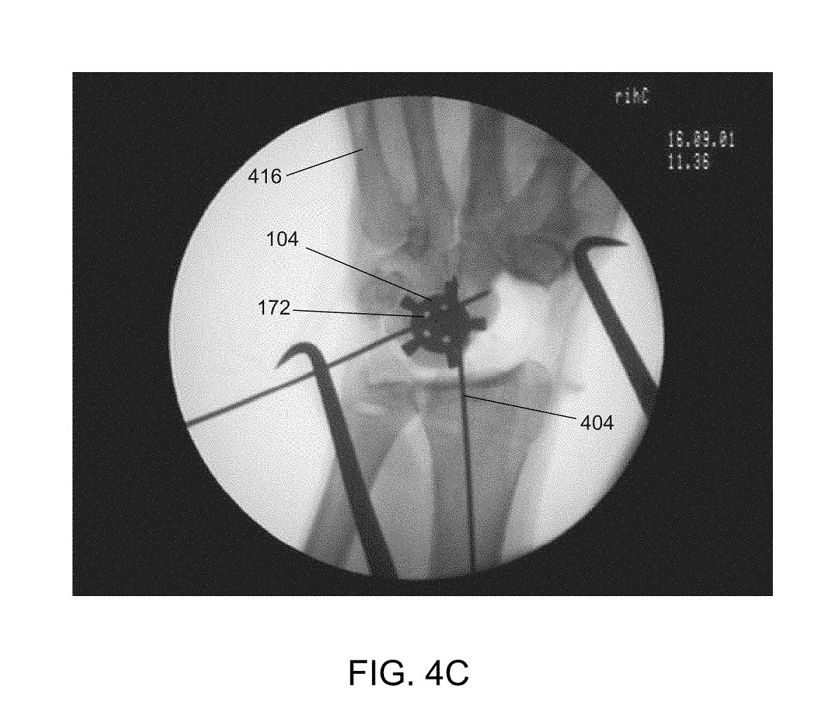

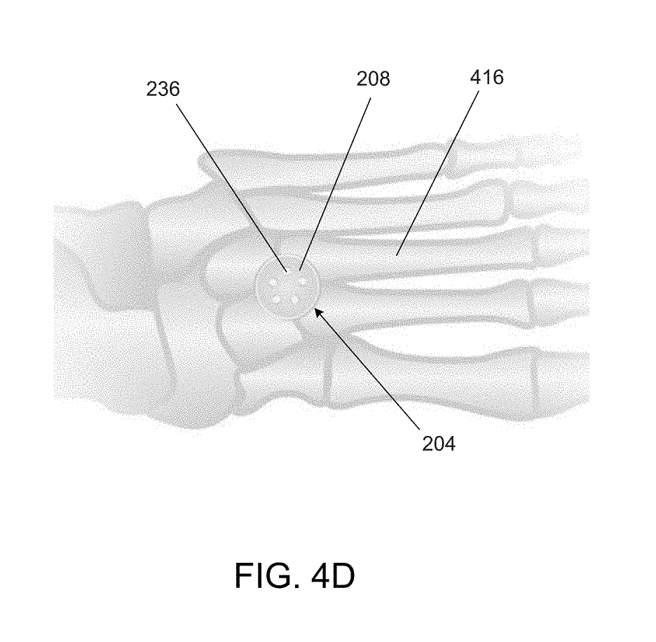

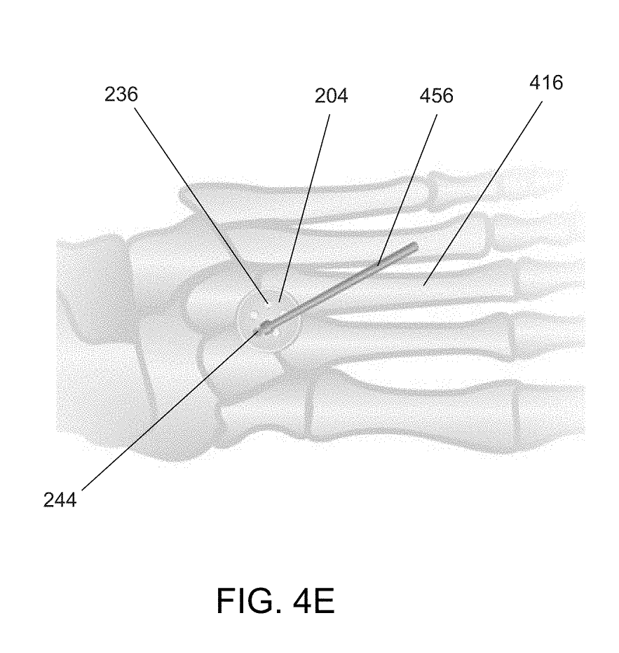

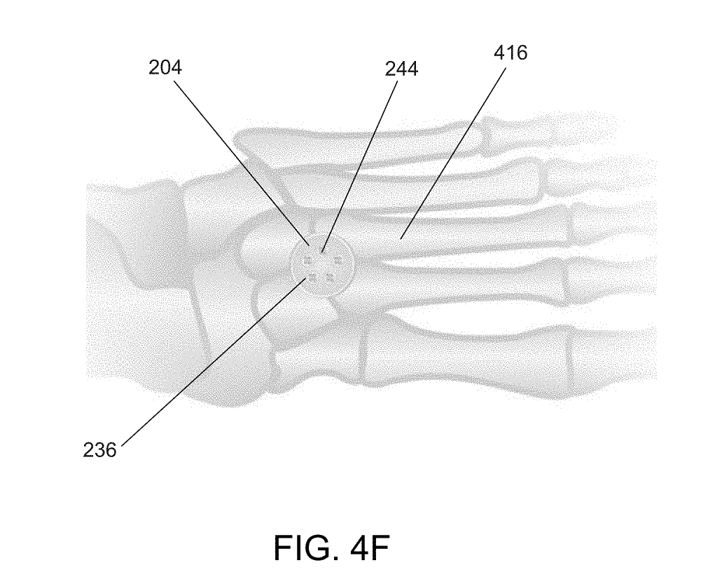

FIGS. 4A-4F depict various views of stages an embodiment 400 of the present methods using guide 100 of FIGS. 1A-1E with implant 200 of FIGS. 2A-2H. As shown in FIG. 4A, a guide pin 404 having a distal end 408 and a proximal end 412 and is inserted into a bone 416 at a position at which implant 200 is intended to be centered. Distal end 408 of guide pin 404 can be sharpened or tapered to improve the ease and/or precision with which guide pin 404 can penetrate bone 416 as shown in FIG. 4A. Guide pin 404 can also have threads, rough surfaces, and/or some other suitable characteristic near the distal end 408 to enable guide pin 404 to penetrate bone 416 and resist inadvertent removal. In the embodiment shown, guide pin 404 is configured to penetrate the surface of bone 416 to a distance suitable to stabilize guide pin 404 in a position along central axis 420. As shown, guide pin 404 has a cylindrical shape. In some embodiments, guide pin 404 is comprised of a radiopaque material.

As shown in FIG. 4B, a reamer 424 is coupled to (e.g., around) guide pin 404 while guide pin 404 remains affixed into bone 416 in the same position as shown, for example, in FIG. 4A. In the embodiment shown, reamer 424 comprises a body 428 that extends outwardly along a rotational axis 448. Body 428 has a proximal end 436 and a distal end 440 with a cutting element 444 disposed at distal end 440. Reamer 424 is configured to be rotated around rotational axis 448 (e.g., in direction 452) with cutting element 444 in contact with bone 416 to remove a portion of one or more bones 416 (e.g., and impart the shape of cutting element 444 to bone 416) to create a recess in bones 416.

Reamer 424 includes an opening at proximal end 436 defining a first end of a hollow cavity in body 428 through which rotational axis 448 extends to permit reamer 424 to be disposed over guide pin 404. A similar opening at distal end 440 defines a second end of the hollow cavity in body 428. A channel extends between these openings such that guide pin 404 can pass through the inside body 428 via the channel to permit reamer 424 to be rotated around guide pin 404. In this embodiment, rotational axis 448 is coaxial with central axis 420 of guide pin 404. In this way, reamer 424 can move along guide pin 404 until distal end 440 contacts bone 416. Reamer 424 can then be rotated to cause cutting element 444 to cut a recess into one or more bones 416, with the recess centered at the point where guide pin 404 is inserted into bone 416. In some embodiments, a height of cutting element 444 corresponds to a desired depth of the recess to be cut, such that the height of cutting element 444 can provide a reference for the desired depth of the recess. In some embodiments, the height of cutting element 444 is the same or greater than height 132 of body 104 and/or height 232 of body 204. Additionally, the profile of cutting element 444 can be adapted to match the shape of at least a portion of lower side 212 of implant body 204, such that the recess formed by cutting element 444 is shaped and sized to receive at least a portion of lower side 112 of guide body 104 and/or at least a portion of lower side 212 of implant body 204. In some configurations, the diameter of cutting element 444 is the same or greater than diameter 128 of body 104 and/or diameter 228 of body 204, such that the formed recess has a sufficient size to receive guide body 104 or implant body 204 into the recess. In the embodiment shown, reamer 424 is removed from guide pin 404 after the desired recess has been cut.

As shown in FIG. 4C, once reamer 424 is removed from guide pin 404, guide body 104 is inserted into the recess cut into the one or more bones 416 by reamer 424. Guide body 104 receives proximal end 412 of guide pin 404 into guide pin hole 196 and enables guide body 104 to slide along central axis 420 from proximal end 412 toward distal end 408 as guide pin 404 moves through guide pin hole 196. In this way, guide body 104 moves along guide pin 404 until guide body 104 is disposed into the recess cut into the one or more bones 416. In some embodiments, guide body 104 is positioned in the recess with lower side 112 in contact with the surface of the bone(s) that define the recess. In some embodiments such as that shown in FIG. 4C where radiopaque markers 136 comprise protrusions, guide body 104 is positioned in the recess to a depth where the lower sides 152 of radiopaque markers 136 abut an outer surface of bone 416. In the embodiment shown, guide body 104 is configured to be rotatable around guide pin 404 relative to the bone 416. Using guide labels 192 as references, guide body 104 is positioned into the recess in a desired configuration.

As shown in FIG. 4C, the radiopaque nature of markers 136 allows the orientation of guide 100 to be determined under imaging (e.g., X-ray). Specifically, as reflected in the X-ray image shown in FIG. 4C, markers 136 each correspond to the location of one of the fastener holes 236 in implant body 204, such that the position of markers 136 indicate the position of fastener holes 236. As such, if an X-ray reveals that markers 136 are not oriented as desired, or are oriented in an undesirable way (e.g., such that a fastener would extend between two bones instead of into a bone), then guide 100 can be rotated around guide pin 404 to orient markers 136 (and thereby fastener holes 236 in a corresponding implant body 204) as desired, such as, for example, such that each fastener hole 236 aligns with a bone or piece of bone rather than between two bones or pieces of bone. In some embodiments or implementations, such as the one shown in FIG. 4C, one or more additional guide pins 404 are inserted into bone 416 through one or more of guide holes 172. In some such embodiments or implementations, each additional guide pin 404 is inserted while the first guide pin 404 is disposed through guide pin hole 196 and remains embedded in bone 416. In some embodiments, the one or more additional guide pins 404 inserted into guide holes 172 assist with maintaining guide body 104 in the desired orientation while a drill bit or other drilling device is inserted into other guide holes 172 to drill holes into bone 416 along central axes 176. Once the desired holes are formed in the underlying bone for some or all of guide holes 176, guide body 104 is removed from the recess by sliding it off a guide pin 404 that extends through one of guide holes 176 or guide pin hole 196. For example, when preparing an implant site to receive implant 200 (which does not include a central guide pin hole), one additional guide pin 404 can be inserted through one of guide holes 172, and the central guide pin 404 can be removed from central guide pin hole 196, such that the additional guide pin 404 can be used to initially align implant body 204 at the site.

In the embodiment shown, the central guide pin 404 is removed prior to positioning implant 200 with lower side 212 in the recess formed in the underlying bone. As shown in FIG. 4D, implant body 204 without guide pin hole 348 is inserted into the recess cut into bone 416. In some embodiments, implant body 204 is positioned in the recess with lower side 212 in contact with the bottom surface of the recess and/or with upper side 208 positioned to be planar with the outer surface of bone 416. If drill holes have been cut into bone 416, body 204 can be positioned so fastener holes 236 align with the drill holes. In some embodiments, body 204 receives proximal end 412 of a guide pin 404 (that was previously inserted into a guide hole 176 of the guide) into a corresponding fastener hole 236 to guide body 204 along the guide pin to the recess previously cut at the prepared implant site in a similar position to that of guide body 104. Alternatively, if an implant (e.g., 300) with a central guide pin hole (e.g., 348) is used, the original guide pin 404 can remain embedded in bone 416 such that body 304 receives proximal end 412 of guide pin 404 into guide pin hole 348 and enables body 304 to slide along central axis 420 from proximal end 412 toward distal end 408 as guide pin 404 moves through guide pin hole 348 (and body 304 slides or otherwise moves along guide pin 404). In this way, body 304 moves along guide pin 404 until body 304 is disposed into the recess cut into the one or more bones 416, similar to the method described above to dispose guide body 104 into the recess. In either approach, if desired, the remaining guide pin 404 can remain until at least one or two fasteners have been inserted through fastener holes 236 and into the underlying bone to secure implant body 204. In some embodiments, fasteners 244 are secured through all other fastener holes 236 before the remaining guide pin 244 is removed. In some instances, once the remaining guide pin 404 is removed, a drill bit or other drill device is used to enlarge and/or otherwise prepare the corresponding hole in the bone to receive a fastener.

As shown in FIG. 4E, a fastener 244 is inserted through each of fastener holes 236 of implant body 204 (e.g., aligned with the corresponding central axis 176 of the fastener hole) and into a corresponding drilled hole aligned with the respective fastener hole 236 to secure body 204 to underlying bone 416. In the embodiment shown, fasteners 244 are secured and tightened using a driver 456 although other suitable devices can be used. As shown in FIG. 4F, when positioned in this way, implant 200, 300 bridges a multiple bones or sections of bone 416 (e.g., between existing bones or across fractures or other seams between pieces of a bone). Fasteners 244 extends into each of the multiple bones or sections of bone 416 and draws them in toward implant 200 or 300. In this way, implant 200, 300 and fasteners 244 stabilize a fracture, injured joint, or surgery site during a healing process.

Fasteners 244, 344 can comprise screws, bolts, or other suitable fastening devices. In some embodiments, fasteners 244, 344 comprise locking screws. In these embodiments, fastener holes 236 comprise one or more locking mechanisms that receive one or more locking screws or fastener and enable the locking screws/fasteners to lock into the locking mechanisms via threads or other suitable means. In some embodiments, locking screws are used when permanent stabilization is desired or when the severity or location of a fracture or injured joint requires a higher degree of stabilization. In other embodiments, non-locking fasteners 244, 344 are used when temporary stabilization is desired or when the severity or location of a fracture or injured joint only requires a lower to moderate degree of stabilization. In some embodiments, both locking and non-locking fasteners 244, 344 are used.

One or more of the elements described above can be included in an orthopedic device or kit that includes one or more alignment guides (e.g., 100), one or more alignment fusion disks (e.g., 200, 300), one or more guide pins (e.g., 404), a reamer (e.g., 424), and one or more fasteners (e.g., 244, 344). Embodiments of the present guides (e.g., 100), fusion disks (e.g., 200, 300), guide pins (e.g., 404), and/or reamers (e.g., 424) can comprise any materials that permit the respective functions described in this disclosure. For example, the present guides, fusion disks, guide pins, and/or reamers can comprise at least one of: a biocompatible metal, stainless steel, 316L stainless steel, titanium, an allograft material, a xenograft material, polymer, and polyphenylsulfone (PPSU) such as Radel®. Some embodiments of the present orthopedic device or kit 500 comprise an embodiment of the present guides (e.g., 100), fusion disks (e.g., 200, 300), guide pins (e.g., 404), reamers (e.g., 424), and/or fasteners 244, 344 and a package within which the guides, fusion disks, guide pins, reamers and/or fasteners are sealed. In some such embodiments, one or more of the elements of the kit are sterile.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices, apparatuses, kits, and methods are not intended to be limited to the particular forms disclosed. Rather, they include all modifications and alternatives falling within the scope of the claims, and embodiments other than the one shown may include some or all of the features of the depicted embodiment. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A guide apparatus comprising:
a guide body having an upper side and an opposing lower side and a vertical central axis, the body defining a guide pin hole extending through the upper and lower sides of the body and configured to receive a guide pin, the body further defining a plurality of guide holes extending through the upper and lower sides of the body and corresponding to the positions of fastener holes in an implant, and the body having a plurality of radiopaque markers corresponding to at least some of the guide holes;
where the orientation of each of the radiopaque markers is substantially fixed relative to the body to indicate a position of a corresponding one of the guide holes;
where each of the plurality of fastener holes is configured to receive a drill bit and/or a guide pin; and
where the body is configured to be temporarily coupled to a bone, via a guide pin extending through the guide pin hole, such that:
the lower side of the body faces the bone;
the upper side of the body faces away from the bone; and
the body is rotatable around the guide pin relative to the bone.

2. The apparatus of claim 1, where the plurality of guide holes each has a central axis that intersects the vertical central axis of the body at a point located at a distance closer to the upper side of the body than the lower side of the body.

3. The apparatus of claim 2, where the vertical central axis of the body extends through the center of the pin hole, and where the body is configured to be temporarily coupled to the bone by the guide pin extending through the guide pin hole into the bone.

4. The apparatus of claim 1, where the upper side comprises a planar area delineated by a perimeter, and the lower side is convex and extends to the perimeter.

5. (canceled)

6. (canceled)

7. The apparatus of claim 1, where the plurality of radiopaque markers comprise a plurality of protrusions, each extending from the guide body such that a radius extending between the vertical central axis of the body and a distal end of the radiopaque marker is greater than a radius between the vertical central axis of the body and the perimeter of the guide body.

8-12. (canceled)

13. The apparatus of claim 1, where the plurality of radiopaque markers each have an upper side and a lower side opposing the upper side, where the upper side and the lower side are each disposed in a plane parallel to and above the upper side of the guide body.

14. (canceled)

15. The apparatus of claim 1, where each of the plurality of radiopaque markers corresponds to one of the plurality of guide holes, each guide hole being disposed between the vertical central axis of the body and its corresponding radiopaque marker.

16-26. (canceled)

27. A bone-implant apparatus comprising:
an implant body having an upper side comprising a planar area and a lower side comprising a convex area, the lower side opposing the upper side and meeting the upper side at a perimeter, the body defining a plurality of fastener holes extending through the upper side and lower side of the body, each of the fastener holes configured to receive a guide pin;
where the body is configured to be coupled to a bone with one or more fasteners extending through one or more of the plurality of fastener holes into the bone such that:
the lower side of the body faces the bone; and
the upper side of the body faces away from the bone.

28. The apparatus of claim 27, where the body has a vertical central axis and the plurality of fastener holes each has a central axis that intersects the vertical central axis.

29-33. (canceled)

34. The apparatus of claim 28, where the body further comprises a guide pin hole disposed at a center of the body and extending through the upper side and lower side of the body, where a central axis of the guide pin hole corresponds to the vertical central axis of the body, and where the body is configured to be temporarily coupled to the bone by the guide pin extending through the guide pin hole into the bone.

35. (canceled)

36. A kit comprising:
the guide apparatus of claim 1 and/or the bone-implant apparatus of claim 27; and
a package within which the guide apparatus and/or bone-implant apparatus is sealed.

37. (canceled)

38. (canceled)

39. A method of modifying a bone, comprising:
forming a recess in the bone with a cutting device;
positioning the lower side of the guide body of the guide apparatus of claim 1 to abut a bottom surface of the recess;
positioning the plurality of radiopaque markers to a desired position; and
inserting guide pins through one or more of the plurality of guide holes of the guide body into the bone.

40. The method of claim 39, further comprising:
inserting, before forming the recess, a guide pin into the bone in a position where the guide pin is configured to pass through a guide pin hole of the guide body.

41. The method of claim 39, further comprising:
inserting, before inserting the guide pins into the plurality of guide holes, a drill mechanism through one or more of the plurality of guide holes of the guide body; and
drilling one or more holes into the bone with the drill mechanism.

42. The method of claim 41, further comprising:
removing the guide body from the guide pins while the guide pins are inserted into the bone; and
disposing at least a portion of the bone-implant apparatus of claim 27 into the recess.

43. (canceled)

44. The method of claim 39, where positioning the plurality of radiopaque markers to a desired position comprises positioning a plurality of radiopaque markers to abut an outer surface of the bone.

45. The method of claim 42, where disposing the bone-implant apparatus into the recess comprises at least one of:
passing a guide pin through a guide pin hole of the bone-implant apparatus; and
passing a guide pin through a fastener hole of the bone-implant apparatus.

46. (canceled)

47. The method of claim 42, further comprising:
inserting a fastener through each of the fastener holes of the bone-implant apparatus and into one of the one or more holes drilled into the bone.

48. (canceled)

49. The method of claim 47, where each fastener extends into a separate bone.

50. The method of claim 40, where:
the cutting device is a reamer having a body extending outwardly relative to a rotational axis, the body having a proximal cutting end, a distal end, and defining a hollow channel extending through the body from the proximal end to the distal end along the rotational axis; and
forming a recess comprises:
disposing the reamer over the guide pin such that the guide pin extends through the hollow channel and the proximal cutting end is in contact with a surface of the bone; and
rotating the reamer around the guide pin to form the recess in the bone.

51. (canceled)

52. (canceled)
